(19) United States
(12) Patent Application Publication
(10) Pub. No.: US 2015/0216695 A1
(43) Pub. Date: Aug. 6, 2015

(54) CATHETER HAVING A SHEATH INCLUDING A WIRE LAYER

(71) Applicants: Ivonne Heumann, Feuerthalen (CH); Anna Marie Haarmann genannt Thiemann, Buesingen (DE); Simon Hildebrand, Rafz (CH)

(72) Inventors: Ivonne Heumann, Feuerthalen (CH); Anna Marie Haarmann genannt Thiemann, Buesingen (DE); Michael Roth, Radolfzell (DE); Simon Hildebrand, Rafz (CH)

(21) Appl. No.: 14/237,345

(22) PCT Filed: Sep. 6, 2012
(86) PCT No.: PCT/EP2012/003748

§ 371 Date: Oct. 28, 2014

(30) Foreign Application Priority Data
Sep. 9, 2011 (DE) 20 2011 105 503.9

(51) Int. Cl. A61F 2/966

(52) U.S. Cl. CPC A61F 2/966; A61M 25/005

(57) ABSTRACT

The invention relates to a catheter (1) for delivery of a self-expanding stent (7) into a body lumen, comprising a shaft (3) having a distal end and a proximal end, the shaft (3) defining a lumen (5) and a stent accommodating portion at its outside for carrying a self-expanding stent (7), a catheter tip (4) arranged at the distal end of the shaft (3), a sheath (6) arranged concentrically to the shaft (3), a pusher coil (8) arranged concentrically in between the shaft (3) and the sheath (6), and proximal to the stent accommodating portion, wherein the sheath (6) comprises an inside layer (22), an outside layer (25), and a wire layer (23) of interwoven wires (26, 27), the wire layer (23) being arranged in between the inside layer (22) and the outside layer (25).

The invention relates to a catheter for delivery of a self-expanding stent into a body lumen.

### BACKGROUND OF THE INVENTION

In medicine, a natural conduit in a body may be locally flow constricted due to disease. During surgeries, a stent may be inserted into this natural conduit, in order to prevent or counteract such a flow constriction. A stent is an artificial tube-like device having meshed walls, the dimension of the stent, in particular the diameter, being expandable once it is positioned in the natural conduit appropriately, e.g. the local restriction. In the expanded shape, the stent holds the natural conduit open to allow the flow of body fluids or the access for surgery.

There is already a variety of stent deployment systems on the market; however, the high performance expectations and the highly sensitive practical use always require a further technical improvement.

### SUMMARY OF THE INVENTION

It is an object of the present invention to provide a catheter for delivery of a self-expanding stent with improved handling characteristics.

This object is solved with a catheter according to the independent claim. Advantageous further developments are subject of the dependent claims.

According to an embodiment of the invention, a catheter for delivery of a self-expanding stent into a body lumen is provided, the catheter comprises a shaft having a distal end and a proximal end, the shaft defining a lumen and a stent accommodating portion at its outside for carrying a self-expanding stent; a catheter tip arranged at the distal end of the shaft; a sheath arranged concentrically to the shaft (which means that the longitudinal center line of the sheath coincides with the longitudinal center line of the shaft); a pusher coil arranged concentrically (which means that the longitudinal center line of the pusher coil coincides with the longitudinal center line of the sheath and the shaft) in between the shaft and the sheath, and proximal to the stent accommodating portion; wherein the sheath comprises an inside layer, an outside layer, and a wire layer of interwoven wires, the wire layer being arranged in between the inside layer and the outside layer. This embodiment demonstrates enhancement in stent placement accuracy. The sheath having the wire layer of interwoven wires (braided sheath) in combination with the pusher coil reduces the elongation of the sheath under force and improves the force transmission during stent deployment. Further, the catheter shows a better push- and crossability while being tracked through the vessel anatomy to the target lesion due to the more direct force transmission with the braid-enhanced sheath. The pusher coil demonstrates an improved kink resistance and high flexibility.

According to a further embodiment of the invention, the wire layer comprises at least eight helically wound wires which are helically surrounding the inside layer, half of them extending in one direction and the other half extending in the counter direction, the helically wound wires extending in the same direction are offset from each other in equal intervals in a circumferential direction of the sheath and form the same angle with the longitudinal direction of the sheath. This embodiment increases the above mentioned advantages.

The catheter can be further improved by designing it such that the wire layer further comprises at least one straight wire extending along the longitudinal direction of the sheath. Due to the at least one straight wire, the sheath demonstrates an even further reduced elongation of the sheath under force and improves the force transmission during stent deployment. The “longitudinal direction of the sheath” can be considered as an imaginary line in parallel to the longitudinal center line of the sheath.

According to a yet further embodiment of the invention, the helically wound wires and/or the at least one straight wire are flattened wires having cross-sections with a width which is larger than a height, wherein the height is extending in a radial direction of the catheter. This enables to reduce the diameter of the catheter.

According to a further embodiment of the invention, the sheath further comprises an intermediate layer being arranged in between the wire layer and the outside layer. This intermediate layer, preferably a polyimide layer, allows to tightly embed the wire layer (wire braid), which prevents the wire braid from movement/elongation. Therefore, the intermediate layer contributes to the shaft elongation-resistance.

According to another embodiment of the invention, the wire layer comprises exactly sixteen of the helically wound wires. This provides even better the above described advantages. The restriction to “exactly sixteen” only refers to the “helically wound wires” and does per se not restrict or exclude the presence of straight wires or other elements.

Beneficially, the catheter is designed such that the wire layer comprises exactly four of the straight wires. This also provides even better the above described advantages. The restriction to “exactly four” only refers to the “straight wires” and does per se not restrict or exclude the presence of helically wound wires or other elements.

According to a further embodiment of the invention, the wire layer consists of sixteen helically wound wires, four straight wires, and optionally attachment means for physically connecting the wires at selected points with each other. The attachment means can be for example adhesive, glue, a welded joint, a soldered joint, etc.

In a further embodiment of the invention, the straight wires are interwoven with the helical wires.

Advantageously, the above advantages can be demonstrated even better in case the sheath consists of the inside layer, the intermediate layer, the outside layer, and the wire layer of interwoven wires, the wire layer being arranged in between the inside layer and the intermediate layer, and optionally attachment means for physically connecting the layers with each other. The attachment means can be for example adhesive, glue, a melted joint, chemical bounds, friction, etc.

Specifically, it can be advantageous when the outside layer is made of thermoplastic elastomer and/or the intermediate layer is made of polyimide.

According to a further embodiment of the invention, the inside layer is made of polytetrafluoroethylene. This embodiment provides the advantages of reduced stent deployment forces and friction forces of the catheter or deployment system. Tests showed that the manufacturing time and the mechanical load on the stent can be reduced and a stent loading of up to 100 mm length is enabled.

According to a yet further embodiment of the invention, the pusher coil is made of stainless steel. Tests showed that the catheter according to the invention can provide a kink resistance of up to 7N, and a flexibility of up to 4.6 N/mm.

Preferably, a proximal portion of the shaft is made of metal and a distal portion of the shaft is made of polymeric material like, e.g. polyetheretherketone, Nylon, Pebax®, polyethylene, polyamide, polyurethane, or polyimide.

In a preferred embodiment, the shaft is made from any of the above-mentioned polymers with a hypotube (metallic tube) disposed about a proximal portion of the polymertube over a length of about 100 to 300 mm to reinforce the shaft at its proximal end.

According to another further development of the invention, the catheter further comprises a radiopaque marker ring element disposed on the shaft at a position proximal to the catheter tip and distal of or at the distal end of the stent accommodating portion, wherein radiopaque marker ring element is connected to the shaft such that their movements are locked by e.g. gluing or swaging.

According to a further embodiment of the invention, the catheter further comprises a radiopaque marker ring element which is provided concentrically to the pusher coil, and which is permanently connected to the distal end of the pusher coil.

Further, the catheter can further comprise a self-expanding stent disposed about the stent accommodation portion and underneath the sheath.

Preferably, the catheter is designed such that the pusher coil, the sheath and the shaft all have a constant diameter along their lengths, and the pusher coil has a constant helix angle along its length.

These and other embodiments are described in more detail with reference to the Figures.

### BRIEF DESCRIPTION OF THE FIGURES

FIG. 1a shows an overview of the catheter according to an embodiment of the invention;

FIG. 1b is an enlarged view of a part of the catheter shown in FIG. 1a, the part being marked with an oval dotted line 2 in FIG. 1a;

FIG. 2a shows the pusher coil of the catheter according to an embodiment of the invention;

FIG. 2b shows a proximal portion of the pusher coil, the portion being marked in FIG. 2a with line 16;

FIG. 2c shows the pusher coil's distal part which is marked in FIG. 2a with line 17;

FIG. 3a shows a cross-sectional view of a sheath of the catheter according to an embodiment of the invention;

FIG. 3b is an enlarged view of the area marked with 21 in the cross-sectional view shown in FIG. 3a, and

FIG. 3c is a cross-sectional side view along line A-A of the sheath of the catheter according to an embodiment of the invention.

### DETAILED DESCRIPTION OF THE INVENTION

In this specification, “distal” refers to an end of an element, which faces away from a user and faces forward, in the direction the catheter is intended to be inserted into a body lumen.

“Proximal” refers of a side of an element, which faces to the user and faces rearward of the direction the catheter is intended to be inserted into a body lumen.

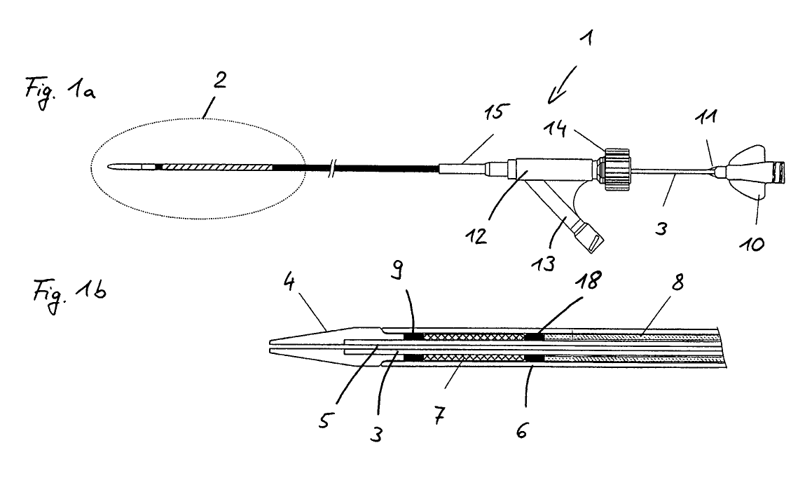

FIG. 1a shows an overview of the catheter 1 according to an embodiment of the invention, and FIG. 1b is an enlarged view of a part of the catheter 1 shown in FIG. 1a, the part being marked with an oval dotted line 2 in FIG. 1a. First, it is mainly referred to FIG. 1b showing more details. The catheter 1 comprises a shaft 3 in form of a tube. Preferably, the proximal part of the catheter 1, preferably the most proximal 20 cm, are made of metal, such as stainless steel, and the remaining part of the catheter (the part distal to the proximal metal part) is made of polyetheretherketone (PEEK), Nylon, Pebax®, polyethylene, polyamide, polyurethane, polyimide or a combination of some of the mentioned materials. This way, the proximal part of the shaft 3 is stiffer than a distal part of the shaft 3. The parts of the shaft 3 having different materials are preferably attached to each other by adhesive bonding. At the distal end of the shaft 3, a catheter tip 4 is mounted on the end of the shaft 3. For this purpose the catheter tip 4 is provided with an additional recess longitudinally reaching from the tip's proximal end to the tip's middle, the recess having an inner diameter substantially corresponding to the outer diameter of the shaft 3, in order to realize a press-fitting or an adhesive attachment between the shaft 3 and the tip 4. In another embodiment, the tip can be injection molded directly onto the distal shaft portion. The outer diameter of the tip 4 is tapered towards the tip's distal end. Both, the shaft 3 and the tip 4 are defining a guide wire lumen 5 along their longitudinal centerlines, wherein the lumen 5 has the same inner diameter within the shaft 3 and the tip 4. Radially outwards and coaxially to the shaft 3, the catheter 1 comprises a sheath 6 in form of a flexible tube. Proximal of the tip 4, the shaft 3 is provided with a stent accommodating portion for placing and carrying a self-expanding stent 7. In an non-retracted position, the sheath 6 abuts the tip 4, wherein an outer diameter of the sheath 6 corresponds to an outer diameter of a middle part of the tip 4. The outer diameter of the proximal end of the tip 4 corresponds to the inner diameter of the sheath 6 such that the distal end of the sheath 6 is tightly slipped over the proximal end of the tip 4 when the sheath 6 is in an extended position. Proximal to the stent accommodating portion, a pusher coil 8 is disposed in a space formed in between the shaft 3 and the sheath 6. In this space, the pusher coil 8 is can be shifted forward (towards the distal end of the catheter 1) or backwards (towards the proximal end of the catheter 1), and shifted independently of the shaft 3 and the sheath 6. Within a space which is bordered by the proximal end of the tip 4, the outside of the shaft 3, the inside of the sheath 6 and the distal end of the pusher coil 8, the stent 7 is accommodated in a non-expanded state when the sheath 6 is in the non-retracted position, in which the distal end of the sheath 6 abuts the tip 4 (as shown in FIG. 1b). In this state, the radially inwards facing side of the sheath 6 is abutting the radially outwards facing side of the stent 7, retaining the stent 7 in its non-expanded state this way. In between the tip 4 and the stent 7, and in between the stent 7 and the pusher coil 8, there is provided a radiopaque marker ring element 9 and 18, respectively, in a state in which the tip 4, the radiopaque marker ring element 9 (first ring element), the stent 7, the radiopaque marker ring element 18 (second ring element) and the pusher coil 8 all abut against each other. The first ring element 9 is adhesively bonded with its side facing radially inwards to the outside of the shaft 3, such that the movement of the first ring element 9 is locked to the movement of the shaft 3. The first ring element 9 is preferably made of tantalum. Thus, starting from the distal end of the catheter 1 proceeding in a proximal direction of the catheter 1, there is provided the tip 4, a first ring element 9, the stent 7, a second ring element 18 and the pusher coil 8, in this order and all directly adjacent to each other. The first ring element 9 has an inner diameter substantially corresponding to the outer diameter of the shaft 3, and an outer diameter substantially corresponding to the inner diameter of the sheath 9. The inner diameter of the pusher coil 8 is slightly larger than the outer diameter of the shaft 3, and the outer diameter of the pusher coil 8 is slightly smaller than the inner diameter of the sheath 6, in order realize a good slideability of the pusher coil 8 in a longitudinal direction of the catheter 1. Alternatively to the above, the catheter 1 can also be realized without the two ring elements 9 and 18 shown in FIG. 1b, but they might be advantageous for determining the positioning of the shaft 3 and the pusher coil 8.

Referring to FIG. 1a, the proximal end of the shaft 3 has a luer lock 10 attached thereto, preferably by means of adhesive 11, preferably light curing glue (UV/VIS glue). The proximal end of the sheath 6 is attached to a distal end of a casing 12 which comprises a luer port 13. The function of the luer lock 10 and the luer port 13 is well known from the state of the art. The casing 12 coaxially accommodates the shaft 3 such that by longitudinal relative movement of the casing 12 along the shaft 3, the sheath 6 is retracted relative to the tip 4 and the shaft 3, such that the stent 7 expands due to the radially outwards acting pre-tension of the self-expanding stent 7 when the sheath 6 is retracted proximally beyond the stent 7. In order to lock or release the relative movement of the shaft 3 and the sheath 6, a lock 14 is provided at the proximal end of the casing 12. By rotating the lock 14 in one direction, a relative movement of the shaft 3 and the sheath 6 can be locked during inserting the catheter 1 into a body lumen, and by rotating the lock 14 in the opposite direction, that relative movement can be released when the stent 7 is in place within the body lumen for being deployed as described above. At the distal end of the casing 12, there is provided a shrinking hose 15 for sealing the sheath/casing attachment.

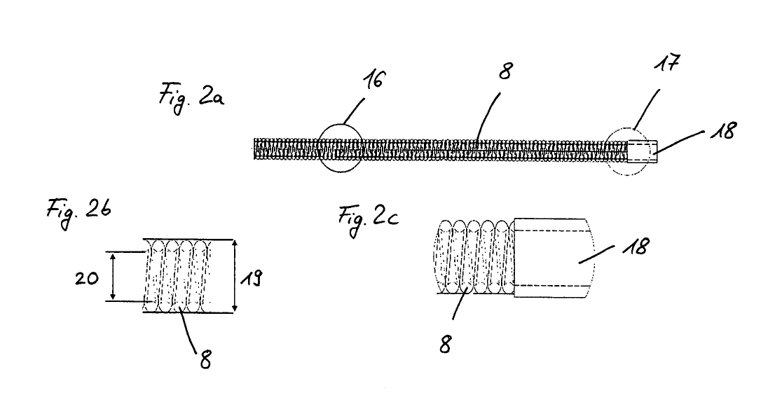

FIG. 2a shows the pusher coil 8 of the catheter 1 according to an embodiment of the invention, FIG. 2b shows a proximal part of the pusher coil which is marked in FIG. 2a with line 16, and FIG. 2c shows the pusher coil's distal part which is marked in FIG. 2a with line 17. As depicted in the Figures, the pusher coil 8 has a constant diameter and helix angle. At the distal end of the pusher coil 8, the second ring element or radiopaque marker band 18 is attached. The pusher coil 8 has preferably a total length of 79 to 135 cm. Further the pusher coil 8 has preferably an outer diameter 19 of 1.05 mm, an inner diameter 20 of 0.77 mm and a helix angle of 2.8°. The helix angle is the constant angle at which any helix of the pusher coil 8 cuts the longitudinal of the pusher coil 8 in an imaginary cylinder formed by the pusher coil 8. The second ring element 18 is either provided coaxially and longitudinally overlapping with the pusher coil 8 such that the proximal end of the pusher coil 8 corresponds in a longitudinal direction of the catheter 1 with the proximal end of the second ring element 18, or coaxially and adjacent to the pusher coil 8 such that the proximal end of the pusher coil 8 abuts the distal end of the second ring element 18. The attachment of the pusher coil 8 and the second ring element 18 is realized by welding, and the second ring element 18 is preferably made of tantalum and has an inner and outer diameter corresponding to the respective dimensions of the pusher coil 8.

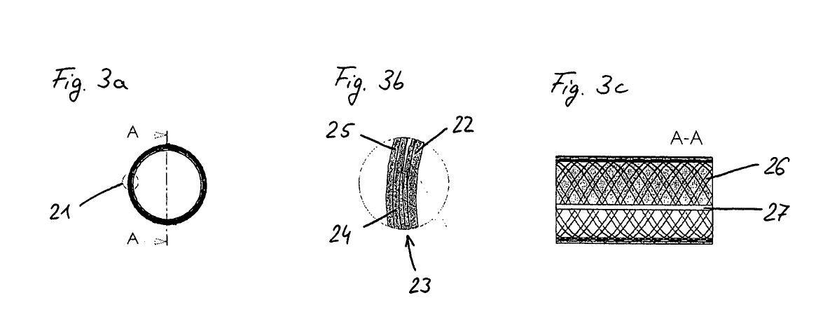

FIG. 3a shows a cross-sectional view of a sheath 6 of the catheter 1 according to an embodiment of the invention; FIG. 3b is an enlarged view of the area marked with 21 in the cross-sectional view shown in FIG. 3a, and FIG. 3c is a cross-sectional side view along line A-A of the sheath 6 of the catheter according to an embodiment of the invention. The sheath is comprising, and preferably composed of, four layers. From the center of the sheath 6 in a radially outwards direction, there is provided an inside layer 22 made of polytetrafluoroethylene (PTFE), a wire layer 23 which is described in more detail below, an intermediate layer 24 made of polyimide, and an outside layer 25 made of thermoplastic elastomer, preferably Pebax® 72D. The four layers 22-25 are concentrical and directly contacting each other. The wire layer 23 comprises, and preferably consists of, sixteen helically wound wires 26 which are helically wound around the outside of the inside layer 22. The helical wires 26 surround the inside layer 22 helically, half of them extending in one direction and the other half extending in the counter direction, such that they form an interwoven braid. The helically wound wires 26 which extend in the same direction form the same angles with the longitudinal direction of the sheath 6, and are offset from each other in a circumferential direction of the sheath 6 by equal intervals. Further, there are provided four straight wires 27 which extend within the wire layer 23 in parallel to the centerline of the sheath 6, and are offset from each other by 90° in a circumferential direction of the sheath 6. The wires are preferably rectangular and made of stainless steel with the dimensions 0.0127×0.0762 mm (0.0005×0.003 inch; 90 ppi count). The stainless steel braid reduces stent embedment during aging. The helical wires and the straight wires are interwoven with each other, in order to form a braid.

While the invention has been illustrated and described in detail in the drawings and foregoing description, such illustration and description are to be considered illustrative or exemplary and not restrictive and it is not intended to limit the invention to the disclosed embodiments. The mere fact that certain measures are recited in mutually different dependent claims does not indicate that a combination of these measures cannot be used advantageously.

1. Catheter (1) for delivery of a self-expanding stent (7) into a body lumen, comprising:
a shaft (3) having a distal end and a proximal end, the shaft (3) defining a lumen (5) and a stent accommodating portion at its outside for carrying a self-expanding stent (7);
a catheter tip (4) arranged at the distal end of the shaft (3);
a sheath (6) arranged concentrically to the shaft (3);
a pusher coil (8) arranged concentrically in between the shaft (3) and the sheath (6), and proximal to the stent accommodating portion;
wherein the sheath (6) comprises an inside layer (22), an outside layer (25), and a wire layer (23) of interwoven wires (26, 27), the wire layer (23) being arranged in between the inside layer (22) and the outside layer (25).

2. Catheter (1) according to claim 1,
wherein the wire layer (23) comprises at least eight helically wound wires (26) which are helically surrounding the inside layer (22), half of them extending in one direction and the other half extending in the counter direction, the helically wound wires (26) extending in the same direction are offset from each other in equal intervals in a circumferential direction of the sheath (6) and form the same angle with the longitudinal direction of the sheath (6).

3. Catheter (1) according to one of the preceding claims,
wherein the wire layer (23) further comprises at least one straight wire (26) extending along the longitudinal direction of the sheath (6).

4. Catheter (1) according to one of the preceding claims,
wherein the helically wound wires (26) and/or the at least one straight wire are flattened wires having cross-sections with a width which is larger than a height, wherein the height is extending in a radial direction of the catheter.

5. Catheter (1) according to one of the preceding claims,
wherein the sheath (6) further comprises an intermediate layer (24) being arranged in between the wire layer (23) and the outside layer (25).

6. Catheter (1) according to one of the preceding claims,
wherein the wire layer (23) comprises exactly sixteen of the helically wound wires (26).

7. Catheter (1) according to one of the preceding claims,
wherein the wire layer (23) comprises exactly four of the straight wires (27).

8. Catheter (1) according to one of the preceding claims,
wherein the wire layer (23) consists of sixteen helically wound wires (26), four straight wires (27), and optionally attachment means for physically connecting the wires at selected points with each other.

9. Catheter (1) according to one of the preceding claims,
wherein the sheath (6) consists of the inside layer (22), the intermediate layer (24), the outside layer (25), and the wire layer (23) of interwoven wires (26, 27), the wire layer (23) being arranged in between the inside layer (22) and the intermediate layer (24), and optionally attachment means for physically connecting the layers with each other.

10. Catheter (1) according to one of the preceding claims,
wherein the outside layer (25) is made of thermoplastic elastomer.

11. Catheter (1) according to claim 5,
wherein the intermediate layer (24) is made of polyimide.

12. Catheter (1) according to one of the preceding claims,
wherein the inside layer (22) is made of polytetrafluoroethylene.

13. Catheter (1) according to one of the preceding claims,
wherein the pusher coil (8) is made of stainless steel.

14. Catheter (1) according to one of the preceding claims,
wherein the shaft (3) is made of polymeric material like polyetheretherketone, Nylon, Pebax®, polyethylene, polyamide, polyurethane, or polyimide and a proximal portion of the shaft (3) is reinforced by a metal tube disposed over the polymeric tube.

15. Catheter (1) according to one of the preceding claims, further comprising a radiopaque marker ring element (9) disposed on the shaft (3) at a position proximal to the catheter tip (4) and distal of or at the distal end of the stent accommodating portion, wherein radiopaque marker ring element is connected to the shaft such that their movements are locked.

16. Catheter (1) according to one of the preceding claims, further comprising a radiopaque marker ring element (18) which is provided concentrically to the pusher coil (8), and which is permanently connected to the distal end of the pusher coil (8).

17. Catheter (1) according to one of the preceding claims, further comprising a self-expanding stent (7).

18. Catheter (1) according to one of the preceding claims, wherein the pusher coil (8), the sheath (6) and the shaft (3) all have a constant diameter along their lengths, and the pusher coil (8) has a constant helix angle along its length.
